(19) United States
(12) Patent Application Publication
(10) Pub. No.: US 2024/0058374 A1
(43) Pub. Date: Feb. 22, 2024

(54) METHODS FOR PREVENTING CANCER RELAPSE

(71) Applicants: Virginia Commonwealth University, Richmond, VA (US); GlycoMira Therapeutics, Inc., Salt Lake City, UT (US); Department of Veterans Affairs, Washington, DC (US)

(72) Inventors: Thomas P. Kennedy, Williamsburg, VA (US); Bhaumik B. Patel, Glen Allen, VA (US)

(21) Appl. No.: 18/264,879

(22) PCT Filed: Feb. 10, 2022
(86) PCT No.: PCT/US22/15905

§ 371 Date: Aug. 9, 2023

Related U.S. Application Data
(60) Provisional application No. 63/148,245, filed on Feb. 11, 2021.

(51) Int. Cl. A61K 31/737; A61K 31/513; A61K 33/243; A61P 35/00

(52) U.S. Cl. CPC A61K 31/737; A61K 31/513; A61K 33/243; A61P 35/00

(57) ABSTRACT

Described herein are methods for preventing a relapse of cancer in a subject. The methods involve administering to the subject in need thereof a sulfated glycosaminoglycan or the pharmaceutically acceptable salt or ester thereof. In one aspect, the methods described herein prevent the growth or self-renewal of cancer stem cells in a subject. In another aspect, the methods described herein kill active or dormant cancer stem cells in a subject. The methods described herein can be used in combination with chemotherapy and/or radiation. The methods described herein are versatile with respect to preventing the relapse of a number of different cancers.

### CROSS-REFERENCE TO RELATED APPLICATIONS

This application claims the benefit of and priority to co-pending U.S. Provisional Patent Application No. 63/148,245, filed on Feb. 11, 2021, the contents of which are incorporated by reference herein in their entireties.

### BACKGROUND

Approximately 17 million Americans were afflicted with cancer in 2019. That number is projected to go up to over 22 million by 2030. A large majority of these patients will suffer from cancer-related morbidity/mortality due to disease relapse several months to years after resection of their original cancer. Almost all of these relapses are due to reawakening of tumor-renewing but dormant cancer stem cells (CSCs) that disseminate early during the disease course and survive chemotherapy and/or radiation treatment. None of the current FDA-approved therapies can reliably eliminate these dormant CSCs protected in their niche. Hence, there is an urgent unmet need for preventing cancer relapse.

### SUMMARY

Described herein are methods for preventing a relapse of cancer in a subject. The methods involve administering to the subject in need thereof a sulfated glycosaminoglycan or the pharmaceutically acceptable salt or ester thereof. In one aspect, the methods described herein prevent the growth or self-renewal of cancer stem cells in a subject. In another aspect, the methods described herein kill active or dormant cancer stem cells in a subject. The methods described herein can be used in combination with chemotherapy and/or radiation. The methods described herein are versatile with respect to preventing the relapse of a number of different cancers.

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows a schematic of the action of GM1111 on dormant cancer stem cells.

FIG. 2 shows the sustained inhibition of self-renewal of colon cancer stem cells by GM-1111.

FIGS. 3A-3C show the efficacy of GM-1111 in a dual cancer stem cell enriched HT-29 xenograft model.

FIGS. 4A-4B show that GM-1111 protects normal stem cells while inhibiting tumor cancer stem cells.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” “having,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of” and “is.”

As used in the specification and the appended claims, the singular forms “a,” “an” and “the” include plural referents unless the context clearly dictates otherwise. Thus, for example, reference to “a solvent” includes, but is not limited to, mixtures or combinations of two or more such solvents, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

“Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or cannot be substituted and that the description includes both unsubstituted lower alkyl and lower alkyl where there is substitution.

The term “alkyl group” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. In one aspect, the alkyl group is a C₁-C₁₀ branched or straight chain alkyl group. In a further aspect, the alkyl group is methyl. The alkyl group can be unsubstituted or substituted. In the case when the alkyl group is substituted, one or more hydrogen atoms present on the alkyl group can be replaced with or more groups including, but not limited to, alkynyl, alkenyl, aryl, nitro, amino, ester, ketone, aldehyde, hydroxy, carboxylic acid, aralkyl, or alkoxy.

The term “fluoroalkyl group” refers to an alkyl group as defined herein wherein one or more hydrogen atoms of the alkyl group are substituted with a fluorine atom. In certain aspects, the fluoroalkyl group includes at least one trifluoromethyl group. In other aspects, the fluoroalkyl group has the formula —CH₂(CF₂)ₙCF₃, wherein n is an integer of 1, 2, 3, 4, 5, 6, 7, 8, 9, or 10. In one aspect, the fluoroalkyl group is —CH₂CF₂CF₃ or —CH₂CF₂CF₂CF₃.

The term “prevent” or “preventing” as used herein is defined as eliminating or reducing the likelihood of the occurrence of one or more symptoms of a disease or disorder (e.g., relapse of cancer) when using the methods as described herein when compared to a control where the method is not used.

As used herein, the term “effective amount” refers to an amount that is sufficient to achieve the desired modification of a physical property of the composition or material. For example, an “effective amount” of a filler in a pharmaceutical composition refers to an amount that is sufficient to achieve the desired improvement in the property modulated by the formulation component, e.g. achieving the desired size or volume for a pharmaceutical dosage form. The specific level in terms of wt % in a composition required as an effective amount will depend upon a variety of factors including the active ingredient, dosage, presence of other therapeutic agents in the dosage form, and intended method of administration of the pharmaceutical composition.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. For example, hyaluronan that contains at least one —OH group can be represented by the formula Y—OH, where Y is the remainder (i.e., residue) of the hyaluronan molecule.

“Subject” refers to mammals including, but not limited to, humans, non-human primates, sheep, dogs, rodents (e.g., mouse, rat, etc.), guinea pigs, cats, rabbits, cows, horses, wildlife (e.g., lions, tigers, etc.) and non-mammals including chickens, amphibians, and reptiles.

The term “relapse of cancer” as used herein is defined as the return of a cancer or the signs and symptoms of a cancer after a period of improvement. Here, the cancer that has recurred after a period of time during which the cancer could not be detected. The cancer may come back to the same place as the original (primary) tumor or to another place in the body.

The term “cancer stem cells” or CSCs as used herein are defined as a small subpopulation of cells within tumors with capabilities of self-renewal, differentiation, and tumorigenicity. Dormant cancer stem cells are viable cells that do not actively proliferate but retain the ability to do so at the opportune time in the future causing cancer relapse. Active cancer stem cells generate cancer cells and subsequent cancer relapse.

Described herein are methods for preventing a relapse of cancer in a subject. The methods involve administering to the subject in need thereof an effective amount of a sulfated glycosaminoglycan or the pharmaceutically acceptable salt or ester thereof. In one aspect, the methods described herein prevent the growth or self-renewal of cancer stem cells in a subject. In another aspect, the methods described herein kill active or dormant cancer stem cells in a subject. The methods described herein can be used in combination with chemotherapy and/or radiation.

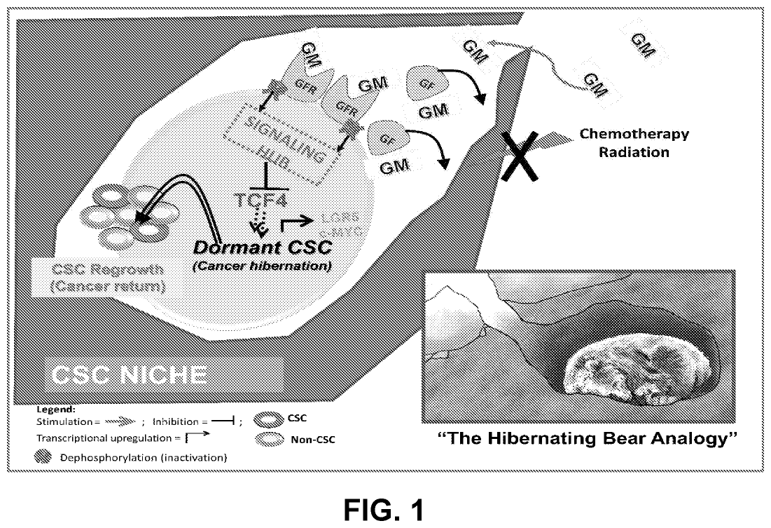

Cancer relapse and spread is caused by the reawakening of cancer stem cells (CSCs) that seed every tumor. Not wishing to be bound by theory, the sulfated glycosaminoglycan can inhibit dormant CSCs and prevent cancer return via inhibition of CSC regrowth. As shown in FIG. 1, CSCs are protected in a niche analogous to a hibernating bear in a cave and hence are off-limits to chemotherapy and radiation. Thus, the use of the sulfated glycosaminoglycans described in combination with chemotherapy and/or radiation treatment is a potent approach for not only treating cancer but also preventing the relapse of cancer.

In one aspect, the sulfated glycosaminoglycans used herein can prevent self-renewal of CSCs. Although inhibition of CSCs following by anti-cancer agents at low concentrations is very encouraging for its potential clinical value, more often than not, anti-cancer agents cannot be continued or tolerated by the patient indefinitely. CSCs possess the ability to self-renew, i.e., generate a copy of itself while generating a rapidly proliferating progenitor. The sulfated glycosaminoglycans described herein interfere with this fundamental process, which results in sustained inhibition of CSCs. In one aspect, the sulfated glycosaminoglycans can inhibit quaternary spheroids three generations after cessation of treatment, which is indicative of reduction of CSC self-renewal. Thus, the use of the sulfated glycosaminoglycans in the methods described herein can produce long-lasting effects beyond the cessation of the drug treatment offering the potential of long-term remission and cure.

The methods described herein can be used in combination with other traditional chemotherapy treatment. In one aspect, the sulfated glycosaminoglycans can be administered concurrently with one or more chemotherapeutic agents and/or radiotherapy. In another aspect, the sulfated glycosaminoglycans can be administered sequentially with one or more chemotherapeutic agents. For example, one or more chemotherapeutic agents can be administered to a subject with cancer in order to reduce tumor size or volume. After treatment with the chemotherapeutic agents, the subject can be administered a sulfated glycosaminoglycan described herein to prevent cancer relapse. While some chemotherapeutic agents may be effective in reducing the size or volume of tumors, they are not effective in inhibiting CSC self-renewal. The methods described herein address this issue and provide a two-prong approach for treating patients with cancer as well as offer long-term remission and cure.

The methods described herein are versatile with respect to preventing the relapse of a number of different cancers and tumor types. In one aspect, the subject has breast cancer, ovarian cancer, lung cancer, kidney cancer, brain cancer, colorectal cancer, neck cancer, head cancer, pancreatic and biliary cancer, prostate cancer, or melanoma. In one aspect, the methods described herein are useful while treating a patient with cancer (e.g., treating a patient with active cancer in combination with chemotherapy and/or radiation treatment). In other aspects, the methods described herein involve administering to the subject a sulfated glycosaminoglycan after the subject has been treated for cancer, where the subject is in remission.

In addition to preventing cancer relapse, the sulfated glycosaminoglycan also protects normal, non-malignant tissue and normal non-malignant stem cells from radiation injury. In another aspect, the sulfated glycosaminoglycans described herein can also protect normal tissue against damage due to chemotherapy. In particular, the sulfated glycosaminoglycans protect non-malignant stem cells of normal tissue. This is a unique aspect, where the viability of cancer stem cells is inhibited while protecting stem cells from normal nonmalignant tissue. This is an important feature because many cancers such as, for example, colorectal, head and neck, esophageal, lung, anal and bladder cancer are treated with regimens incorporating cancer focused radiation, which unfortunately can harm surrounding normal tissue despite best attempts to specifically direct most radiation to the tumor itself. Thus, the sulfated glycosaminoglycan can protect normal tissue/stem-cells against radiation while eliminating CSC is highly desirable for organ preservation approaches. Patients with aerodigestive and genitourinary cancers, including but not limited to colorectal, head and neck, esophageal cancer, lung cancer, anal carcinoma, bladder cancer, etc. will be able to keep their cancer afflicted organs intact and fully functional upon administration of the sulfated glycosaminoglycan along with chemotherapy and/or radiation treatment. Ultimately, the methods described herein provide a robust improvement in cancer patients' quality of life as well as their functional capacity.

The sulfated glycosaminoglycan is a glycosaminoglycan (GAG) having at least one sulfate group. Generically, GAGs are represented by the formula A-B-A-B-A-B, where A is an uronic acid and B is an aminosugar that is either O- or N-sulfated, where the A and B units can be heterogeneous with respect to epimeric content or sulfation. In one aspect, the sulfated glycosaminoglycan is chondroitin sulfate, dermatan sulfate, or heparan sulfate.

In one aspect, the sulfated glycosaminoglycan is a sulfated hyaluronan or the pharmaceutically acceptable salt or ester thereof. In one aspect, the sulfated hyaluronan has a degree of sulfation from 0.1 to 4.0 per disaccharide unit. In another aspect, the sulfated hyaluronan has a degree of sulfation from 0.1, 0.5, 1.0, 1.5, 2.0, 2.5, 3.0, 3.1, 3.2, 3.3, 3.4, 3.5, 3.6, 3.7, 3.8, 3.9, or 4.0 per disaccharide unit, where any value can be a lower and upper end-point of a range (e.g., 3.0 to 4.0, 3.2 to 3.8, etc.).

In another aspect, the average molecular weight of the sulfated hyaluronan is less than 1,000 kDa, less than 900 kDa, less than 800 kDa, less than 700 kDa, less than 600 kDa, less than 500 kDa, less than 400 kDa, less than 300 kDa, less than 200 kDa, less than 100 kDa, less than 50 kDa, less than 25 kDa, less than 10 kDa, or less than 5 kDa. In another aspect, the sulfated hyaluronan has an average molecular size from 0.5 kDa to less than 50 kDa, 2 Da to 20 kDa, or 3 kDa to 10 kDa. In a further aspect, the sulfated hyaluronan has an average molecular size from 0.5 kDa to 10 kDa or 1 kDa to 10 kDa. Depending upon reaction conditions, one or more different hydroxyl groups present in the low molecular hyaluronan or hyaluronan oligosaccharide can be sulfated. In one aspect, the primary C-6 hydroxyl proton of the N-acetyl-glucosamine residue of the low molecular hyaluronan or hyaluronan oligosaccharide is sulfated. In another aspect, the primary C-6 hydroxyl proton of the N-acetyl-glucosamine residue of hyaluronan and at least one C-2 hydroxyl proton or C-3 hydroxyl proton of a uronic acid residue or at least one C-4 hydroxyl proton of an N-acetyl-glucosamine residue is substituted with a sulfate group. In another aspect, the primary C-6 hydroxyl proton of the N-acetyl-glucosamine residue of the low molecular hyaluronan or hyaluronan oligosaccharide and at least one C-2 hydroxyl proton and C-3 hydroxyl proton of a uronic acid residue and at least one C-4 hydroxyl proton of an N-acetyl-glucosamine residue is substituted with a sulfate group. In another aspect, 0.001%, 0.01%, 0.1%, 1%, 5%, 10%, 20%, 30%, 40%, 50%, 60%, 70%, 80%, 90%, 95%, or less than 100%, or any range thereof of hydroxyl protons present on the low molecular hyaluronan or hyaluronan oligosaccharide can be deprotonated and subsequently sulfated.

In another aspect, the sulfated hyaluronan has (1) 90% of the primary C-6 hydroxyl protons of the N-acetyl-glucosamine residue of the sulfated hyaluronan are substituted with a sulfate group, (2) a degree of sulfation from 3.0 to 4.0, and (3) an average molecular weight from 1 kDa to 10 kDa. In another aspect, sulfated hyaluronan has (1) 100% of the primary C-6 hydroxyl protons of the N-acetyl-glucosamine residue of the sulfated hyaluronan are substituted with a sulfate group, (2) a degree of sulfation from 3.0 to 4.0, and (3) an average molecular weight from 1 kDa to 10 kDa.

The hyaluronan starting material used to produce the sulfated hyaluronan can exist as the free acid or the salt thereof. Derivatives of hyaluronan starting material can also be used herein. The derivatives include any modification of the hyaluronan prior to and/or after sulfation. A wide variety of molecular weight hyaluronans can be used herein for the depolymerization step. In one aspect, the hyaluronan has a molecular weight greater than 1,000 kDa prior to depolymerization. In another aspect, the hyaluronan can have a molecular weight of 10 kDa to 1,000 kDa prior to depolymerization. A wide variety of hyaluronan molecular weights can also be employed for the sulfation step. In one aspect, the hyaluronan starting material can be converted to low molecular hyaluronan or a hyaluronan oligosaccharide prior to sulfation to produce the partially or fully sulfated hyaluronan. As will be discussed in greater detail below, low molecular weight hyaluronan is hyaluronan that has been degraded with an acid or base or depolymerized by techniques known in the art including, but not limited to, ultrasound, ozonolysis, sheer stress, or radical-mediated chain cleavage. Alternatively, hyaluronan oligosaccharide is produced by degrading hyaluronan with an enzyme such as, for example, hyaluronan synthase or hyaluronidase in a controlled fashion. Subsequently, hyaluronan oligosaccharides having different molecular weights can be separated by GPC or ion exchange separation. Exemplary procedures for producing low molecular weight hyaluronan or hyaluronan oligosaccharide from hyaluronan are provided in WO 2011/156445.

In one aspect, the low molecular hyaluronan or hyaluronan oligosaccharide being sulfated has a molecular weight from 1 kDa to 2,000 kDa. In another aspect, the low molecular hyaluronan or hyaluronan oligosaccharide being sulfated has a molecular weight from 5 kDa to 500 kDa, 10 kDa to 200 kDa, or 20 kDa to 100 kDa, or less than 200 kDa, 150 kDa, 100 kDa, 75 kDa, 50 kDa, or 20 kDa. Exemplary procedures for preparing low molecular weight hyaluronan are provided in WO 2011/156445. As discussed above, the molecular weight of the hyaluronan can be modified by cleaving hyaluronan with an acid or base to produce lower molecular weight hyaluronan. In certain aspects, the hyaluronan starting material or a derivative thereof is not derived from an animal source. In these aspects, the hyaluronan can be derived from other sources such as bacteria. For example, a recombinant B. subtilis expression system can be used to produce the hyaluronan starting material.

After the low molecular hyaluronan or hyaluronan oligosaccharide has been treated with a base, it is reacted with a sulfating agent to produce the partially or fully sulfated hyaluronan. Sulfating agents commonly used in organic synthesis can be used herein. Examples of sulfating agents include, but are not limited to, pyridine-sulfur trioxide complex, chlorosulfonic acid, or the triethylamine-sulfur trioxide complex. In one aspect, low molecular hyaluronan or hyaluronan oligosaccharide can be converted to the tributylamine salt, lyophilized, resuspended in dimethylformamide, and subsequently treated with a sulfating agent (e.g., pyridine-sulfur trioxide complex) to sulfate one or more hydroxyl protons.

In one aspect, when the sulfating agent is a pyridine-sulfur trioxide complex, a pyridinium adduct of the sulfated hyaluronan is produced, where pyridine is covalently attached to the sulfated hyaluronan. Not wishing to be bound by theory, when hyaluronan is reacted with the pyridine-sulfur trioxide complex in a solvent such as, for example, DMF, a small amount of acid is produced from traces of water present in situ, which causes partial depolymerization resulting in a free reducing end group. The hydroxyl group of the hemiketal can ultimately be sulfated to produce a sulfated intermediate, which subsequently reacts with free pyridine produced in situ to produce the pyridinium adduct. Thus, the sulfated hyaluronan used herein can include a mixture of sulfated hyaluronan that does not have pyridine covalently attached to the molecule and sulfated hyaluronan that does have pyridine covalently attached to the molecule. In one aspect, from 0.01% to 100%, 0.1% to 10%, or 0.15% to 2.5% of the sulfated hyaluronan has pyridine covalently attached to the molecule. In another aspect, the molecular weight of the pyridinium adduct of the sulfated hyaluronan is less than or equal to 10 kDa. In other aspects, the molecular weight is 0.1 kDa, 0.5 kDa, 1 kDa, 2 kDa, 3 kDa, 4 kDa, 5 kDa, 6 kDa, 7 kDa, 8 kDa, 9 kDa, or 10 kDa, where any value can for the lower and upper end-point of a molecular weight range.

In another aspect, the sulfated glycosaminoglycan is hyaluronan or its pharmaceutically acceptable salt or ester having at least one sulfate group and at least one primary C-6 hydroxyl position of an N-acetyl-glucosamine residue comprising an alkyl group or fluoroalkyl group.

In one aspect, at least one primary C-6 hydroxyl proton of the N-acetyl-glucosamine residue of hyaluronan is substituted with an alkyl group. In another aspect, at least one primary C-6 hydroxyl proton of the N-acetyl-glucosamine residue of hyaluronan is substituted with a fluoroalkyl group. In another aspect, the methylated/sulfated hyaluronan has the formula depicted below:

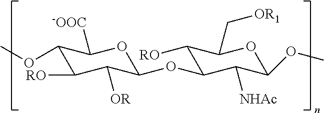

where R₁ is a methyl group, while the remaining R groups are sulfate groups alone or in combination with hydrogen. In one aspect, n is from 5 to 20, 5 to 15, 5 to 10, or 7 to 9.

In another aspect, the sulfated glycosaminoglycan can be a mixture composed of a first methylated/sulfated hyaluronan and a second methylated/sulfated hyaluronan with pyridine covalently bonded to the methylated/sulfated hyaluronan can be used in the methods described herein.

In one aspect, the mixture includes
(a) a first modified hyaluronan or a pharmaceutically acceptable salt or ester thereof, wherein said first modified hyaluronan or its pharmaceutically acceptable salt or ester comprises (i) at least one primary C-6 hydroxyl proton of at least one N-acetyl-glucosamine residue substituted with a methyl group, (ii) an average molecular weight from 1 kDa to 15 kDa, (iii) a degree of methylation greater than 0 to 0.5 methyl groups per disaccharide unit; and (iv) a degree of sulfation of 2.5 to 4.0 sulfate groups per disaccharide unit; and
(b) a second modified hyaluronan or a pharmaceutically acceptable salt or ester thereof, wherein said second modified hyaluronan or its pharmaceutically acceptable salt or ester comprises (i) at least one primary C-6 hydroxyl proton of at least one N-acetyl-glucosamine residue substituted with a methyl group, (ii) an average molecular weight from 1 kDa to 15 kDa, (iii) a degree of methylation greater than 0 to 0.5 methyl groups per disaccharide unit; and a (iv) degree of sulfation of 2.5 to 4.0 sulfate groups per disaccharide unit, wherein pyridine is covalently bonded to the second modified hyaluronan or a pharmaceutically acceptable salt or ester thereof.

In one aspect, the degree of methylation in the first and second modified hyaluronan is 0.030, 0.050, 0.075, 0.100, 0.125, 0.150, 0.175, 0.200, 0.225, 0.250, 0.275, 0.300, 0.325, 0.350, 0.375, 0.400, 0.425, 0.45, 0.475, or 0.500 methyl groups per disaccharide unit, where any value can be a lower and upper endpoint of a range (e.g., 0.030 to 0.300, 0.100 to 0.200, etc.). In one aspect, only the primary C-6 hydroxyl proton of an N-acetyl-glucosamine residue of the first and second modified hyaluronan is substituted with the methyl group (i.e., methyl group is only at this position). In other aspects, 1% to 100% 5% to 100%, 10% to 100%, 20% to 100%, 50% to 100%, 60% to 100%, 70% to 100%, 80% to 100%, 90% to 100%, or 95% to 100% of the primary C-6 hydroxyl protons of the N-acetyl-glucosamine residue of the first and second modified hyaluronan are replaced with a methyl group.

In another aspect, the first and second modified hyaluronan have an average molecular weight 1 kDa, 2 kDa, 3 kDa, 4 kDa, 5 kDa, 6 kDa, 7 kDa, 8 kDa, 9 kDa, 10 kDa, 11 kDa, 12 kDa, 13 kDa, 14 kDa, or 15 kDa, where any value can be a lower and upper endpoint of a range (e.g., 1 kDa to 10 kDa, 3 kDa to 7 kDa, etc.).

In another aspect, the first and second modified hyaluronan have a degree of sulfation of 2.5, 2.75, 3.00, 3.25, 3.50, 3.75, or 4.00 sulfate groups per disaccharide unit, where any value can be a lower and upper endpoint of a range (e.g., 1.5 to 3.5, 3. to 4.0, etc.).

In another aspect, the amount of pyridine in the mixture of the first and second modified hyaluronan is 0.10, 0.25, 0.50, 0.75, 1.00, 1.25, 1.50, 1.75, 2.00, 2.25, 2.50, 2.75, 3.00, 3.25, 3.50, 3.75, or 4.00 wt % of the mixture, where any value can be a lower and upper endpoint of a range (e.g., 0.500 to 3.00, 1.00 to 2.00, etc.). The amount of pyridine can be quantified by ¹H NMR and UV spectroscopy.

In another aspect, the degree of methylation in the first and second modified hyaluronan is 0.03 to 0.3 methyl groups per disaccharide unit, the first and second modified hyaluronan has an average molecular weight from 1 kDa to 10 kDa, the degree of sulfation in the first and second modified hyaluronan is 3.0 to 4.0 sulfate groups per disaccharide unit, and the amount of pyridine present in the composition is from 0.1 wt % to 4.0 wt % of the composition.

The hyaluronan starting material can exist as the free acid or the salt thereof. Derivatives of hyaluronan starting material can also be used herein. The derivatives include any modification of the hyaluronan prior to the alkylation or fluoroalkylation step. A wide variety of molecular weight hyaluronan can be used herein. In one aspect, the hyaluronan has a molecular weight greater than 10 kDa prior to alkylation or fluoroalkylation. In another aspect, the hyaluronan has a molecular weight from 25 kDa to 1,000 kDa, 100 kDa to 1,000 kDa, 25 kDa to 500 kDa, 25 kDa to 250 kDa, or 25 kDa to 100 kDa prior to alkylation or fluoroalkylation. In certain aspects, the hyaluronan starting material or a derivative thereof is not derived from an animal source. In these aspects, the hyaluronan can be derived from other sources such as bacteria. For example, a recombinant B. subtilis expression system can be used to produce the hyaluronan starting material.

The hyaluronan starting material or derivative thereof is initially reacted with a sufficient amount of base to deprotonate at least one primary C-6 hydroxyl proton of the N-acetyl-glucosamine residue. The selection of the base can vary. For example, an alkali hydroxide such as sodium hydroxide or potassium hydroxide can be used herein. The concentration or amount of base can vary depending upon the desired degree of alkylation or fluoroalkylation. In one aspect, the amount of base is sufficient to deprotonate at least 0.001% of the primary C-6 hydroxyl protons of the N-acetyl-glucosamine residue of the hyaluronan starting material or derivative thereof. In other aspects, the amount of base is sufficient to deprotonate from 0.001% to 50%, 1% to 50% 5% to 45%, 5% to 40%, 5% to 30%, 5% to 20%, 10% to 50%, 20% to 50%, or 30% to 50% of the primary C-6 hydroxyl protons of the N-acetyl-glucosamine residue of the hyaluronan starting material or derivative thereof. It is understood that the more basic the solution, the more likely are chain cleavage reactions and the higher the degree of alkylation/fluoroalkylation that can be achieved. For example, other hydroxyl groups present on hyaluronan (e.g., 2-OH and/or 3-OH can be alkylated or fluoroalkylated). In one aspect, all of the hydroxyl groups present on hyaluronan can be alkylated or fluoroalkylated. In other aspects, 0.001%, 0.01%, 0.1%, 1%, 5%, 10%, 20%, 30%, 40%, 50%, 60%, 70%, 80%, 90%, 95%, 100%, or any range thereof of hydroxyl protons present on hyaluronan can be deprotonated and subsequently alkylated or fluoroalkylated.

After the hyaluronan starting material or derivative thereof has been treated with a base, the deprotonated hyaluronan is reacted with an alkylating agent or fluoroalkylating agent to produce the sulfated glycosaminoglycan. Examples of alkylating agents include, but are not limited to, an alkyl halide. Alkyl bromides and iodides are particularly useful. Similarly, the fluoroalkylating agent can include a fluoroalkyl halide. Alkylating agents and fluoroalkylating agents commonly used in organic synthesis can be used herein.

In certain aspects, it is desirable to sulfate the alkylated or fluoroalkylated hyaluronan described above. In one aspect, the alkylated or fluoroalkylated hyaluronan is sulfated by reacting the alkylated or fluoroalkylated SAGE with a sulfating agent to produce a sulfated product. The degree of sulfation can vary from partial sulfation to complete sulfation. In general, free hydroxyl groups present on the alkylated or fluoroalkylated hyaluronan or a derivative thereof can be sulfated. In one aspect, at least one C-2 hydroxyl proton and/or C-3 hydroxyl proton is substituted with a sulfate group. In another aspect, the degree of sulfation is from 0.5, 1.0, 1.5, 2.0, 2.5, 3.0, 3.5 or any range thereof per disaccharide unit of the alkylated or fluoroalkylated hyaluronan. In one aspect, the alkylated or fluoroalkylated SAGE can be treated with a base to deprotonate one or more hydroxyl protons followed by the addition of the sulfating agent. The sulfating agent is any compound that reacts with a hydroxyl group or deprotonated hydroxyl group to produce a sulfate group. The molecular weight of the hyaluronan can vary depending upon reaction conditions. In one aspect, the molecular weight of the SAGE is from 2 kDa to 500 kDa, 2 kDa to 250 kDa, 2 kDa to 100 kDa, 2 kDa to 50 kDa, 2 kDa to 25 kDa, or from 2 kDa to 10 kDa.

In one aspect, the alkyl group of the SAGE is methyl and at least one C-2 hydroxyl proton and/or C-3 hydroxyl proton of hyaluronan is substituted with a sulfate group. In another aspect, the alkyl group of the SAGE is methyl, at least one C-2 hydroxyl proton and/or C-3 hydroxyl proton of hyaluronan is substituted with a sulfate group, and the compound has a molecular weight of 2 kDa to 200 kDa after alkylation.

Any of the sulfated and alkylated/fluoroalkylated hyaluronan useful herein can be the pharmaceutically acceptable salt or ester thereof. Pharmaceutically acceptable salts are prepared by treating the free acid with an appropriate amount of a pharmaceutically acceptable base. Representative pharmaceutically acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like. In one aspect, the reaction is conducted in water, alone or in combination with an inert, water-miscible organic solvent, at a temperature of from about 0° C. to about 100° C. such as at room temperature. The molar ratio of compounds of structural formula I to base used are chosen to provide the ratio desired for any particular salts. For preparing, for example, the ammonium salts of the free acid starting material, the starting material can be treated with approximately one equivalent of pharmaceutically acceptable base to yield a neutral salt.

Ester derivatives are typically prepared as precursors to the acid form of the compounds—as illustrated in the examples below—and accordingly can serve as prodrugs. Generally, these derivatives will be lower alkyl esters such as methyl, ethyl, and the like. Amide derivatives —(CO)NH₂, —(CO)NHR and —(CO)NR₂, where R is an alkyl group defined above, can be prepared by reaction of the carboxylic acid-containing compound with ammonia or a substituted amine. Also, the esters can be fatty acid esters. For example, the palmitic ester has been prepared and can be used as an alternative esterase-activated prodrug.

The sulfated glycosaminoglycan described herein can be formulated in any excipient the biological system or entity can tolerate to produce pharmaceutical compositions. Examples of such excipients include, but are not limited to, water, aqueous hyaluronic acid, saline, Ringer's solution, dextrose solution, Hank's solution, and other aqueous physiologically balanced salt solutions. Nonaqueous vehicles, such as fixed oils, vegetable oils such as olive oil and sesame oil, triglycerides, propylene glycol, polyethylene glycol, and injectable organic esters such as ethyl oleate can also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosol, cresols, formalin and benzyl alcohol. In certain aspects, the pH can be modified depending upon the mode of administration. For example, the pH of the composition is from about 5 to about 6, which is suitable for topical applications. Additionally, the pharmaceutical compositions can include carriers, thickeners, diluents, preservatives, surface active agents and the like in addition to the compounds described herein.

The pharmaceutical compositions can also include one or more active ingredients used in combination with the sulfated glycosaminoglycan described herein. The resulting pharmaceutical composition can provide a system for sustained, continuous delivery of drugs and other biologically-active agents to tissues adjacent to or distant from the application site. The biologically-active agent is capable of providing a local or systemic biological, physiological or therapeutic effect in the biological system to which it is applied. For example, the agent can act to control and/or prevent infection or inflammation, enhance cell growth and tissue regeneration, control tumor growth, act as an analgesic, promote anti-cell attachment, reduce alveolar bone and tooth loss, inhibit degeneration of cartilage and weight bearing joints, and enhance bone growth, among other functions. Additionally, any of the compounds described herein can contain combinations of two or more pharmaceutically-acceptable compounds. Examples of such compounds include, but are not limited to, antimicrobial agents, anti-inflammatory agents, anesthetics, and the like. Methods for using these compositions as drug delivery devices is described in detail below.

The pharmaceutical compositions can be prepared using techniques known in the art. In one aspect, the composition is prepared by admixing a sulfated glycosaminoglycan with a pharmaceutically-acceptable compound and/or carrier. The term “admixing” is defined as mixing the two components together so that there is no chemical reaction or physical interaction. The term “admixing” also includes the chemical reaction or physical interaction between the compound and the pharmaceutically-acceptable compound. Covalent bonding to reactive therapeutic drugs, e.g., those having nucleophilic groups, can be undertaken on the compound. Second, non-covalent entrapment of a pharmacologically active agent in a cross-linked polysaccharide is also possible. Third, electrostatic or hydrophobic interactions can facilitate retention of a pharmaceutically-acceptable compound in the compounds described herein.

The sulfated glycosaminoglycan can be administered in a number of ways depending on whether local or systemic treatment is desired, and on the area to be treated. Administration can be topically (including ophthalmically, vaginally, rectally, intranasally, orally, buccally, otologically, or directly to the skin or a mucosal membrane). Formulations for topical administration can include ointments, lotions, creams, gels, drops, suppositories, sprays, liquids and powders. Conventional pharmaceutical carriers, aqueous, powder or oily bases, thickeners and the like can be necessary or desirable. Administration can also be directly into the lung by inhalation of an aerosol or dry micronized powder.

The sulfated glycosaminoglycan can also be injected parenterally either intravenously, subcutaneously, intramuscularly, intradermally, intranasally, intrathecally, subdermally, or by inhalation. In other aspects, the sulfated glycosaminoglycan is administered rectally by an enema, suppository, catheter, needleless syringe, or bulb syringe. In another aspect, the sulfated glycosaminoglycan is formulated as an aerosol, micronized powder, spray, wash, lavage, or other suitable formulations typically used in nasal applications or administration by inhalation. In another aspect, the sulfated glycosaminoglycan or the pharmaceutically acceptable salt or ester thereof is administered intratumorally using techniques known in the art.

It will be appreciated that the actual preferred amounts of the sulfated glycosaminoglycan in a specified case will vary according to the specific compound being utilized, the particular compositions formulated, the mode of application, and the particular situs and subject being treated. Dosages for a given host can be determined using conventional considerations, e.g. by customary comparison of the differential activities of the subject compounds and of a known agent, e.g., by means of an appropriate conventional pharmacological protocol. Physicians and formulators, skilled in the art of determining doses of pharmaceutical compounds, will have no problems determining dose according to standard recommendations (Physicians Desk Reference, Barnhart Publishing (1999). For example, when administered intravenously the dosage of the sulfated glycosaminoglycan can be from 1 mg/kg to 500 mg/kg. In another aspect, when administered orally the dosage of the sulfated glycosaminoglycan can be from 500 mg/kg to 3,000 mg/kg. In another aspect, when administered topically the dosage of the sulfated glycosaminoglycan can be from 1% w/v to 20% w/v. In another aspect, the sulfated glycosaminoglycan or a pharmaceutically acceptable salt or ester thereof is administered to the subject in the amount of 0.1 mg/kg to 500 mg/kg per single dose, 3 mg/kg to 300 mg/kg per single dose, or 10 mg/kg to 100 mg/kg per single dose.

Depending upon the nature of the cancer and treatment schedule, the sulfated glycosaminoglycan can be administered over different intervals of time. In one aspect, when the subject has cancer, the sulfated glycosaminoglycan can be administered to the subject prior to cancer treatment (e.g., chemotherapy and/or radiation), concurrently with cancer treatment, post cancer treatment, or any combination thereof.

The sulfated glycosaminoglycan or the pharmaceutically acceptable salt or ester thereof can be administered once a day or multiple times per day (e.g., 2×, 4×, 8× daily or every other day) before, during, and/or after cancer treatment. The sulfated glycosaminoglycan can be administered over a period of time during and after cancer treatment. In one aspect, the sulfated glycosaminoglycan or the pharmaceutically acceptable salt or ester thereof is administered to the subject daily for up to 28 days after cancer treatment. In another aspect, the sulfated glycosaminoglycan or a pharmaceutically acceptable salt or ester thereof is administered to the subject daily or every other day for 2 days, 3 days, 4 days, 5 days, 6 days, 7 days, 8 days, 9 days, 10 days, 12 days, 14 days, 16 days, 18 days, 20 days, 22, days, 24 days, 26 days, or 28 days after cancer treatment, where any value can be a lower and upper-endpoint of a range (e.g., 2 days to 8 days).

### Aspects

Aspect 1. A method for preventing a relapse of cancer in a subject, the method comprising administering to the subject in need thereof a sulfated glycosaminoglycan or the pharmaceutically acceptable salt or ester thereof.

Aspect 2. A method for preventing the growth or self-renewal of cancer stem cells in a subject, the method comprising administering to the subject in need thereof a sulfated glycosaminoglycan or the pharmaceutically acceptable salt or ester thereof.

Aspect 3. A method for killing active cancer stem cells, dormant cancer stem cells, or a combination thereof in a subject, the method comprising administering to the subject in need thereof a sulfated glycosaminoglycan or the pharmaceutically acceptable salt or ester thereof.

Aspect 4. The method of any one of Aspects 1 to 3, wherein the sulfated glycosaminoglycan or the pharmaceutically acceptable salt or ester thereof prevents the formation of quaternary spheroids three generations after the administration of the sulfated glycosaminoglycan or the pharmaceutically acceptable salt or ester thereof.

Aspect 5. The method of any one of Aspects 1 to 3, wherein the sulfated glycosaminoglycan or the pharmaceutically acceptable salt or ester thereof active kills cancer stem cells, dormant cancer stem cells, or a combination thereof in a subject.

Aspect 6. The method of any one of Aspects 1 to 5, wherein the subject was previously treated for cancer.

Aspect 7. The method of any one of Aspects 1 to 5, wherein the subject is being treated for cancer.

Aspect 8. The method of any one of Aspects 1 to 7, wherein the subject is further treated with chemotherapy, radiotherapy, or a combination thereof.

Aspect 9. The method of any one of Aspects 1 to 8, wherein the subject has breast cancer, ovarian cancer, lung cancer, kidney cancer, brain cancer, colorectal cancer, neck cancer, head cancer, pancreatic and biliary cancer, prostate cancer or melanoma.

Aspect 10. The method of any one of Aspects 1 to 9, wherein the sulfated glycosaminoglycan or a pharmaceutically acceptable salt or ester thereof comprises a sulfated hyaluronan or the pharmaceutically acceptable salt or ester thereof.

Aspect 11. The method of Aspect 10, wherein at least one primary C-6 hydroxyl proton of the N-acetyl-glucosamine residue is substituted with a sulfate group.

Aspect 12. The method of Aspect 10, wherein from 1% to 100% of the primary C-6 hydroxyl protons of the N-acetyl-glucosamine residue of hyaluronan are substituted with a sulfate group.

Aspect 13. The method of Aspect 10, wherein at least one C-2 hydroxyl proton and C-3 hydroxyl proton of a uronic acid residue and at least one C-4 hydroxyl proton of an N-acetyl-glucosamine residue is substituted with a sulfate group.

Aspect 14. The method of Aspect 10, wherein the compound has a degree of sulfation from 0.1 to 4.0 per disaccharide unit.

Aspect 15. The method of Aspect 10, wherein the sulfated hyaluronan has an average molecular size of less than 200 kDa.

Aspect 16. The method of Aspect 10, wherein the sulfated hyaluronan has an average molecular size of less than 20 kDa.

Aspect 17. The method of Aspect 10, wherein the sulfated hyaluronan has an average molecular size from 1 kDa to 10 kDa.

Aspect 18. The method of Aspect 17, wherein (1) greater than 90% of the primary C-6 hydroxyl protons of the N-acetyl-glucosamine residue of the sulfated hyaluronan are substituted with a sulfate group, and (2) the sulfated hyaluronan has a degree of sulfation from 3.0 to 4.0.

Aspect 19. The method of Aspect 17, wherein (1) 100% of the primary C-6 hydroxyl protons of the N-acetyl-glucosamine residue of the sulfated hyaluronan are substituted with a sulfate group, and (2) the sulfated hyaluronan has a degree of sulfation from 3.0 to 4.0.

Aspect 20. The method of Aspect 10, wherein the pharmaceutically acceptable ester is a prodrug.

Aspect 21. The method of any one of Aspects 1 to 9, wherein the sulfated glycosaminoglycan or the pharmaceutically acceptable salt or ester comprises at least one sulfate group and at least one primary C-6 hydroxyl position of an N-acetyl-glucosamine residue comprising an alkyl group or fluoroalkyl group.

Aspect 22. The method of Aspect 21, wherein the alkyl groups is an unsubstituted alkyl group.

Aspect 23. The method of Aspect 21, wherein the unsubstituted alkyl group is methyl.

Aspect 24. The method of Aspect 21, wherein the fluoroalkyl group comprises at least one trifluoromethyl group.

Aspect 25. The method of Aspect 21, wherein from 1% to 100% of the primary C-6 hydroxyl protons of the N-acetyl-glucosamine residue are substituted with an alkyl group or fluoroalkyl group.

Aspect 26. The method of Aspect 21, wherein the modified hyaluronan has a molecular weight from 10 kDa to 2,000 kDa prior to modification.

Aspect 27. The method of Aspect 21, wherein at least one C-2 hydroxyl proton and C-3 hydroxyl proton is substituted with a sulfate group.

Aspect 28. The method of Aspect 21, wherein the modified hyaluronan is sulfated at the C-4 hydroxyl position of the N-acetyl glucosamine moiety, the C-2 position of the glucuronic acid moiety, the C-3 position of the glucuronic acid, or any combination thereof.

Aspect 29. The method of Aspect 21, wherein the modified hyaluronan has a degree of sulfation from 0.5 to 4.0 per disaccharide unit.

Aspect 30. The method of Aspect 21, wherein the alkyl group is methyl and at least one C-2 hydroxyl proton and/or C-3 hydroxyl proton is substituted with a sulfate group.

Aspect 31. The method of Aspect 21, wherein the alkyl group is methyl, at least one C-2 hydroxyl proton and/or C-3 hydroxyl proton is substituted with a sulfate group, and the compound has a molecular weight of 2 kDa to 10 kDa.

Aspect 32. The method of Aspect 21, wherein the sulfated glycosaminoglycan comprises
(a) a first modified hyaluronan or a pharmaceutically acceptable salt or ester thereof, wherein said first modified hyaluronan or its pharmaceutically acceptable salt or ester comprises (i) at least one primary C-6 hydroxyl proton of at least one N-acetyl-glucosamine residue substituted with a methyl group, (ii) an average molecular weight from 1 kDa to 15 kDa, (iii) a degree of methylation greater than 0 to 0.5 methyl groups per disaccharide unit; and (iv) a degree of sulfation of 2.5 to 4.0 sulfate groups per disaccharide unit; and
(b) second modified hyaluronan or a pharmaceutically acceptable salt or ester thereof, wherein said second modified hyaluronan or its pharmaceutically acceptable salt or ester comprises (i) at least one primary C-6 hydroxyl proton of at least one N-acetyl-glucosamine residue substituted with a methyl group, (ii) an average molecular weight from 1 kDa to 15 kDa, (iii) a degree of methylation greater than 0 to 0.5 methyl groups per disaccharide unit; and a (iv) degree of sulfation of 2.5 to 4.0 sulfate groups per disaccharide unit, wherein pyridine is covalently bonded to the second modified hyaluronan or a pharmaceutically acceptable salt or ester thereof.

Aspect 33. The method of Aspect 32, wherein the degree of methylation in the first and second modified hyaluronan is 0.03 to 0.3 methyl groups per disaccharide unit.

Aspect 34. The method of Aspect 32, wherein the first and second modified hyaluronan has an average molecular weight from 1 kDa to 10 kDa.

Aspect 35. The method of Aspect 32, wherein the degree of sulfation in the first and second modified hyaluronan is 3.0 to 4.0 sulfate groups per disaccharide unit.

Aspect 36. The method of Aspect 32, wherein the amount of pyridine present is from 0.1 wt % to 4.0 wt % of the composition.

Aspect 37. The method of Aspect 32, the degree of methylation in the first and second modified hyaluronan is 0.03 to 0.3 alkyl groups per disaccharide unit, the first and second modified hyaluronan has an average molecular weight from 1 kDa to 10 kDa, the degree of sulfation in the first and second modified hyaluronan is 3.0 to 4.0 sulfate groups per disaccharide unit, and the amount of pyridine present is from 0.1 wt % to 4.0 wt % of the composition.

Aspect 38. The method of any one of Aspects 1 to 37, wherein the pharmaceutically acceptable salt comprises an organic salt, a metal salt, or a combination thereof.

Aspect 39. The method of Aspect 38, wherein the pharmaceutically acceptable salt of the comprises a salt selected from the group consisting of NH₄⁺, Na⁺, Li⁺, K⁺, Ca⁺², Mg⁺², Fe⁺², Fe⁺³, Cu⁺², Al⁺³, Zn⁺², 2-trimethylethanolammonium cation (choline), or a quaternary salt of isopropylamine, trimethylamine, diethylamine, triethylamine, tripropylamine, ethanolamine, 2-dimethylaminoethanol, 2-diethylaminoethanol, lysine, arginine, and histidine.

Aspect 40. The method of any one of Aspects 1 to 9, wherein the sulfated glycosaminoglycan comprises heparin sulfate or chondroitin sulfate.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process.

### Preparation of Low Molecular Weight Hyaluronan

1. Slowly dissolve 20 g of 850 kDa HA (1% w/v) into 1.7 L of ddH₂O while vigorously stirring over heat (−40° C.). When all 20 g of HA is added, remove from heat and stir until cooled to room temperature, then slowly add 333 mL 6N HCl while stirring. Stir at room temperature for approximately 2 weeks.
2. Use HPLC, GPC, or SEC to monitor degradation reaction at 14 days. Neutralize each sample before analysis to stop reaction and analysis by UV detection at 232 nm, comparing to previous batches of methylated/sulfated hyaluronan.
3. At the molecular weight range of 3-5 kDa, neutralize the reaction to pH 7.0 by slowly adding 40% (w/v) NaOH over ice.
4. Dialyze in 1000 MWCO dialysis tubing against ddH₂O for 24 hrs, changing the water every 6 hrs to obtain hyaluronan fragments of greater than 1 kDa.
5. Lyophilize to obtain a white, fluffy solid. Yield: 12.032 g, 60.2%

### Preparation of Methylated Hyaluronan

1. Dissolve 6.0 g (4% w/v HA in NaOH solution) of low molecular weight hyaluronan in 150 mL of a 40% w/v solution of NaOH in ddH₂O and stir the mixture for 2 hours at room temperature, which generates a viscous solution.
2. Add 225 mL of isopropanol and continue stirring.
3. Add 6 mL (6 eq) of iodomethane and stir the mixture for 24 hours at room temperature.
4. After 24 hours, use a separation funnel to remove the organic solvent layer from the viscous aqueous layer, and add 300 mL of ddH₂O to dilute the crude methylated hyaluronan.
5. Adjust the solution to pH 7.0 with 6N HCl on ice.
6. Allow the neutralized solution to warm to room temperature, and add 3 L of MeOH:EtOH (1:2 v/v) while stirring to precipitate the methylated hyaluronan intermediate. Collect the product by filtration and dry it in a vacuum oven.

### Sulfation of Methylated Hyaluronan to Produce GM-1111

1. Add 2.5 g of crude methylated hyaluronan to 200 mL of anhydrous DMF and stir for 1 h prior to adding 1.56 mL of tributylamine (1 eq). Stir the solution for 20 min at room temperature.
2. Add 25 g of pyridine-sulfur trioxide (24 eq.) by adding 5 g at a time.
3. Stir the mixture for 3 h at 40° C.
4. Cool the reaction on ice and add 50 mL of ddH₂O to quench the reaction.
5. Precipitate the crude material by adding 250 mL of cold 95% ethanol saturated with anhydrous sodium acetate.
6. Centrifuge the crude product at 4,500 rpm for 5-10 min and decant the liquid to collect the light brown gummy solid.
7 Dissolve the crude product in ddH₂O, and dialyze against 20 L of 100 mM NaCl, changing the solution four times a day over 24 h, followed by dialysis against 20 L of distilled water 4 times over 24 h.
8. Lyophilize the dialyzed material. Final Yield: 42.0% of methylated/sulfated hyaluronan (GM-1111)
9. The methylated/sulfated hyaluronan had the following characteristics: average molecular weight is 3 kDa to 7 kDa; average methyl groups per disaccharide unit is 0.3 to 0.3; average degree of sulfation of 3.0 to 4.0; and average pyridine content is 0.1 to 4.0 wt % (pyridine content used in experiments below is 0.69 wt %).

### Example 1. Inhibition of CSC Proliferation

A tandem two-step approach to identify selective anti-CSC agents (PMID: 25325978) was developed. Cells grown as suspended spheroids have more CSCs as opposed to sheets of cells on plastic. Drugs that preferentially inhibit spheroids were investigated. In the second step, it was determined if spheroid growth inhibition is maintained in the second and third generations of spheroid without any further drug treatment. If an agent passes both screens, they are labeled selective anti-CSC agents. Previously, it was discovered that natural and synthesized sugars with sulfur are anti-CSC agents (PMID: 24968014, 27705927, 30337351). Studies in both cell cultures and animals bearing tumors validated the selective inhibition of CSCs, leading to suppression of cancer growth and cancer return. However, some of these early agents had lower potency against CSCs.

Cell Culture: Human colorectal (HT29. WiDr, CaCo2, HT-29, HCT15, SW620, RKO, LOVO, and HCT116) representative of all 4 consensus molecular subtypes of colon cancer as well as SF268 (Brain), MDA MB-231 (Breast), NCI-H460 (Lung), 786-0 (Renal) cells were obtained from NCI or ATCC within 24 months of conducting experiments. These cells were maintained as monolayer in Dulbecco's Modified Eagle Medium: Nutrient Mixture F-12 (DMEM/F-12) supplemented with 10% fetal bovine serum (FBS), and 1% streptomycin/penicillin (AA). The cells were passaged using trypsin containing ethylenediaminetetraacetic acid (EDTA) before they reached 70% confluence.

Spheroid formation: For 1° colonosphere formation, primary cells maintained as monolayer were plated in non-treated, low adhesion, 96 well plates at the concentration of 100-250 cells/100 μL/well in stem cell media (SCM) to generate approximately 20-30 spheroid per well. The SCM consisted of DMEM: F12: AA, supplemented with B27, 20 ng/mL epidermal growth factors and 10 ng/mL fibroblast growth factor. The wells were treated with vehicle or GM-1111 (0→1 mM at half-log concentration increments) at the time of plating. Between days 5-7, numbers of spheres ranging from 50-150 mm in diameter were counted using phase contrast microscope and percent inhibition was calculated compared to vehicle control. IC50 values were calculated with GraphPad Prism using three parametric sigmoidal dose response curve fitting (R²>0.90).

Results: Tables 1 and 2 show that that the concentration required to inhibit 50% of the spheroids (enriched in CSCs) is less than 20 μM for almost all cell colorectal (Table 1) and other CSCs (Table 2) tested. The concentrations at or higher levels can be easily and safely achieved in animals following subcutaneous injection. Importantly, the inhibition of colon CSCs by GM1111 is more seen across various genetic variants and molecular subtypes, suggesting targeting a fundamental process (e.g., signaling hub), as shown in FIG. 1, governing CSC growth.

TABLE 1
GM-1111 inhibits colon CSCs at all four CMS subtypes.
Molecular-Genetic Characteristics

| | | | | | | GM1111 |
|---|---|---|---|---|---|---|
| Cell Line | CMS | p53 | KRAS | BRAF | PIK3CA | (IC₅₀ μM) |
| LOVO^{c} | 1 | WT | G13D | Silent mut | WT | 336 |
| RKO^{a} | 4 | ?WT | WT | V600E | H1047R | 18 |
| SW620ª | 4 | R273H | G12V | WT | WT | 13 |
| HCT15ª | 1 | S241F | G13D | WT | E545K | 8.8 |
| HT-29ª | 3 | R273H | WT | V600E | p449T | 4.8 |
| CaCo2^{b} | 4 | | | | | 0.00425 |
| HCT-116^{b} | 4 | WT | G13D | WT | H1047R | 0.004 |
| WiDr^{b} | 3 | R273H | WT | V600E | p449T | 0.001 |

CMS: consensus molecular subtype
^{a}cells sensitive to GM1111
^{b}cells that are hypersensitive to inhibition to GM1111
^{c}cells resistant to GM1111

TABLE 2
Screening of GM1111 against other CSCs. Other CSCs from
brain, breast, lung, and kidney show sensitivity to GM1111.

| | | Genetic Alteration | | | | GM1111 |
|---|---|---|---|---|---|---|
| Cell | Tissue | p53 | KRAS | BRAF | PIK3CA | (IC₅₀ μM) |
| SF268 | Brain | R273H | WT | WT | WT | 1.9 |
| MB-231 | Breast | R280K | G13D | G464V | WT | 5.3 |
| NCI-H460 | Lung | WT | Q61H | WT | E545K | 1.9 |
| 786-O | Kidney | | | | | 3.8 |

It has been previously reported that specifically sized natural and synthetic hexasaccharide sequences are inhibitors of CSCs (PMID: 27705927). The average molecular weight of such hexasaccharides is approximately 1,000 Da to 1,200 Da. It was discovered that larger molecular weight glycosaminoglycans lose their CSC inhibitory activity. The average molecular weight of GM-1111 is much higher (approximately 4,500 Da). Contrary to the prediction (PMID: 27705927), GM-1111 is several log-fold more potent than even the hexasacchride sequence PMID: 27705927 in inhibiting CSCs. Thus, GM-1111 provides potent CSC growth inhibition despite being much larger than the size of hexasaccharides found previously active against CSC growth. This finding of CSC inhibition despite substantially larger size (approximately four to five-fold larger than previously documented optimum glycosaminoglycan inhibitors of CSC) is an unexpected result based upon previous research.

In summary, GM-1111 potently inhibits colorectal, brain, breast, lung, and kidney CSCs. GM-1111 is effective across tumors of various molecular subtypes and/or carrying different genetic alterations.

### Example 2. Inhibition of CSC Self-Renewal

Methods: HCT-116 colon cancer cells were grown as spheroids in SCM as above and treated with 10 and 30 nM GM-1111 (spanning maximum IC50 concentration observed in the panel of colon CSCs) at the time of plating and primary) (1° spheroids growth inhibition was assessed after 5-7 days (as above). Subsequently, GM-1111 treatment was removed. The primary spheroids were disintegrated into a single cell suspension and propagated (100 cells/well) into secondary (2°) spheroids in SCM as described above for primary spheroids but without further drug exposure. The process was repeated for the tertiary (3°) cultures. numbers of spheres ranging from 50-150 mm in diameter were counted using phase contrast microscope and percent inhibition was calculated compared to vehicle control.

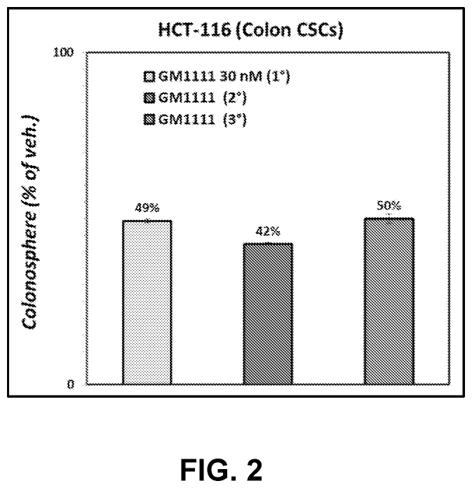

Results: The bar graphs in FIG. 2 show sustained inhibition 50% in 1° →3° despite no further treatment with GM-1111. Hence, GM-1111 not only inhibited HCT-116 primary (1°) colon cancer spheroid growth but, more importantly, once the drug-treatment was stopped, GM-1111 treated spheroids continued to show a robust inhibition in secondary (2°) and tertiary (3°) cultures despite the absence of the drug. In summary, GM-1111 not only inhibits CSC proliferation but also inhibits self-renewal.

### Example 3. Inhibition of CSCs in Chemotherapy-enriched Xenograft Model

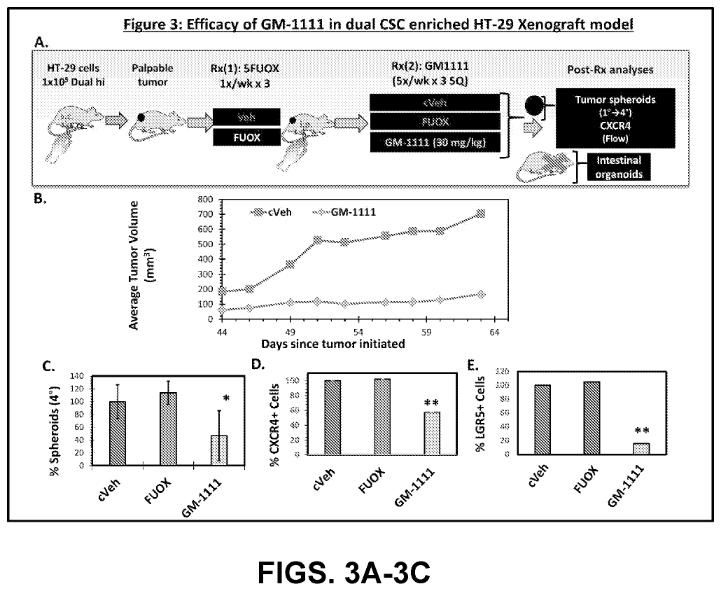

Methods: All experiments involving animals were approved by animal component of the research protocol (ACORP) according to VA McGuire Medical Center (Richmond, VA) guideline). FIG. 3A depicts the schematics. A dual enrichment strategy was utilized. In the first step, in vitro enrichment was performed as depicted in FIG. 3A by developing Dual hi (CD133+/CXCR4+) HT29 cells-derived xenografts in nude mice. Briefly, NCr nude mice were injected with 10⁵ Dual hi HT-29 colon CSCs. Combination of 5-fluorouracil and oxaliplatin (FUOX), which is the most frequently used chemotherapy for colon cancer, enriches CSCs in vitro. A second step of CSC enrichment was performed in vivo. Once the palpable tumors formed (average>50 mm 3), oxaliplatin and 5-fluorouracil (FUOX) was administered, which is the most common colon cancer chemotherapy combination (i.p., weekly for 3 weeks). This strategy has shown to enrich CSCs in xenograft earlier (Boothello R S et al. Mol Cancer Ther. 2019 18:51-61). At the end of first round of FUOX, animals were randomized to vehicle (cVeh), FUOX, or GM-1111 (30 mg/kg SQ five times a week×3 weeks) (N=6-7). Tumor measurements were made three times a week with Vernier calipers, and tumor volume was calculated using the formula: V=W²×(L)/2, where V=volume in mm³, W and L=width and length in mm. The animals were sacrificed at the end of total 6 weeks of treatment.

Animals were sacrificed per IACUC approved methods of euthanasia. The tumor tissue was finely chopped and digested with 400 μg/ml Collagenase Type IV. Single cell suspension was filtered with 70 μm cell strainer (BD Biosciences, San Jose, CA) and were subjected to 1) spheroid formation (1°→4) as above and 2) flow cytometry for CXCR4 and LGR5. Briefly, Xenograft-derived single cells were incubated with conjugated antibody for 30 minutes at 4° C. and washed once with PBS buffer prior to analysis. Following antibody and dilution were used: CXCR4 (anti-mouse CD184)-PE conjugated clone 21311 (Dilution 1:50), and Anti-LGR5 mouse mAb, clone 2A2, PE conjugated (Dilution 1:50). Cell sorting was performed using FACS Aria™ II High-Speed Cell Sorter (BD Biosciences, San Jose, CA) and data were analyzed using FCS express 4 flow research edition.

Results: At the end of 6 weeks of the treatment, the mice treated with GM-1111 showed significant tumor growth inhibition (FIG. 3B). Notably, tumors were analyzed for inhibition of CSCs (spheroids and CXCR4+ cells by flow) [Drug on-target effects]. Indeed, GM-1111 treatment significantly inhibited quaternary spheroids, which is indicative of CSC self-renewal (FIG. 3C) as the growth inhibition persisted despite removal of treatment for several weeks. Additionally, proportion of CXCR4+ cells as well as LGR5⁺ cells (both colonic CSC markers) by >40% compared to cVeh controls (FIG. 3D). Interestingly, animals treated with continued FUOX treatment showed no inhibition of CXCR4+ or LGR5+ cells despite a reduction in tumor volume (not shown),

In summary, GM-1111 and not FUOX treatment not only inhibited CSC-enriched tumor growth but depletes them of CSCs. Furthermore, GM-1111 and not FUOX treatment inhibited CSC self-renewal in vivo. Hence, GM-1111 inhibited drug-resistant CSC population in colorectal cancer.

### Example 4. Safety Evaluation of GM-1111

Methods: Mice were weighed three times a week. At the time of sacrifice, the blood was collected in red-top tube using cardiac puncture. The serum was subjected for chemistry analyses.

Ex-vivo Intestinal organoid generation: Post sacrifice mice intestines were collected in ice cold DMEM/F12 media. The intestines were then flushed in ice-cold PBS using a syringe, following which the intestines were dissected open lengthwise and chopped into 2-4 cm pieces in ice-cold PBS and placed in a 15 mL conical tube. Briefly, these were then subjected to chelation and dissociation into crypts. Approximately 200-500 crypts were then resuspended in 100 μL of matrigel per well of a 96-well plate. The 96-well plate was placed in a CO₂ incubator (37° C., 5% CO₂) for 20 minutes to allow a complete polymerization of the matrigel. Once polymerized, the matrigel was overlayed with 100 μL of IntestiCult™ Organoid Growth Medium (Mouse). The plates were cultured in the CO₂ incubator (37° C., 5% CO₂). The intestinal organoids develop after 5-10 days of culturing.

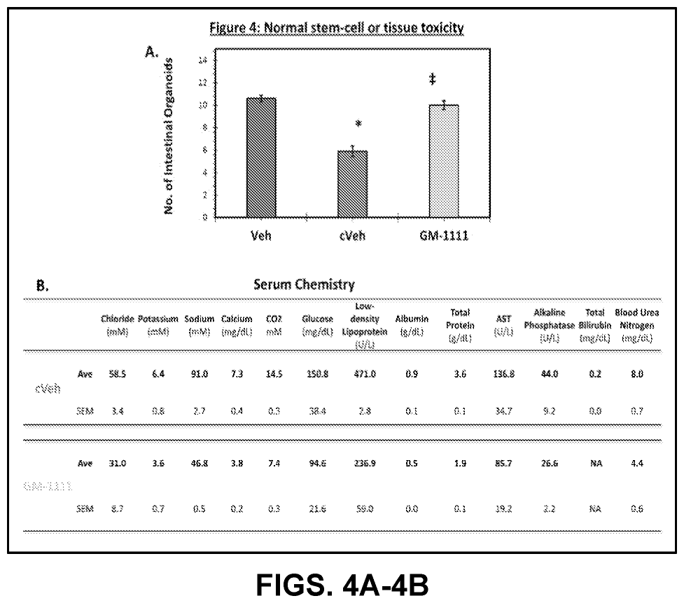

Results: Mice showed normal behavior with no external signs of distress, allergy-induced rashes, or diarrhea. The animal weight also remined stable (no >5% change) throughout the treatment with GM-1111. Serum chemistry studies displayed no significant alterations in serum electrolyte levels or major serum biomarkers indicating hepatic, renal and muscle damage (FIG. 4B). To further understand the effects of GM-1111 on proliferative function of NSCs, ex vivo intestinal organoids were examined. In a striking contrast to the inhibitory effects on the CSCs, GM-1111's effect on NSCs was precisely the opposite. Intestinal epithelial crypts collected from animals treated with FUOX followed by vehicle (cVeh) showed a significant reduction in normal intestinal organoids compared to animals that only received vehicle (Veh) treatment from the study initiation (FIG. 4A). The results suggest toxicity of chemotherapy towards normal intestinal stem/progenitor cells. Interestingly, GM-1111 treatment almost completely rescued the intestinal epithelial cells from the chemotherapy-induced toxicity (FIG. 4A). Hence, GM-1111 not only inhibits tumor CSCs but may also protects normal stem cells.

In summary, GM-1111 is extremely well tolerated at doses that are predicted to results in plasma concentration higher than average IC50 observed in most cells (Tables 1 and 2), while FUOX chemotherapy is toxic towards NSCs. GM-1111 treatment almost completely rescued the intestinal epithelial cells from the chemotherapy-induced toxicity. Hence, GM-1111 represents a unique agent that not only inhibit CSCs but protect normal tissue from damaged caused by common cancer treatments.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

1. A method for preventing a relapse of cancer in a subject, the method comprising administering to the subject in need thereof a sulfated glycosaminoglycan or the pharmaceutically acceptable salt or ester thereof.

2. The method of claim 1, wherein the sulfated glycosaminoglycan or the pharmaceutically acceptable salt or ester thereof prevents the growth or self-renewal of cancer stem cells in a subject.

3. The method of claim 1, wherein the sulfated glycosaminoglycan or the pharmaceutically acceptable salt or ester thereof prevents the formation of quaternary spheroids three generations after the administration of the sulfated glycosaminoglycan or the pharmaceutically acceptable salt or ester thereof.

4. The method of claim 1, wherein the sulfated glycosaminoglycan or the pharmaceutically acceptable salt or ester thereof active kills cancer stem cells, dormant cancer stem cells, or a combination thereof in a subject.

5. The method of claim 1, wherein the subject was previously treated for cancer.

6. The method of claim 1, wherein the subject is being treated for cancer.

7. The method of claim 1, wherein the subject is further treated with chemotherapy, radiotherapy, or a combination thereof.

8. The method of claim 1, wherein the subject has breast cancer, ovarian cancer, lung cancer, kidney cancer, brain cancer, colorectal cancer, neck cancer, head cancer, pancreatic and biliary cancer, prostate cancer or melanoma.

9. The method of claim 1, wherein the subject has colorectal cancer.

10. The method of any one of claims 1 to 9, wherein the sulfated glycosaminoglycan or a pharmaceutically acceptable salt or ester thereof comprises a sulfated hyaluronan or the pharmaceutically acceptable salt or ester thereof.

11. The method of claim 10, wherein at least one primary C-6 hydroxyl proton of the N-acetyl-glucosamine residue is substituted with a sulfate group.

12. The method of claim 10, wherein from 1% to 100% of the primary C-6 hydroxyl protons of the N-acetyl-glucosamine residue of hyaluronan are substituted with a sulfate group.

13. The method of claim 10, wherein at least one C-2 hydroxyl proton and C-3 hydroxyl proton of a uronic acid residue and at least one C-4 hydroxyl proton of an N-acetyl-glucosamine residue is substituted with a sulfate group.

14. The method of claim 10, wherein the compound has a degree of sulfation from 0.1 to 4.0 per disaccharide unit.

15. The method of claim 10, wherein the sulfated hyaluronan has an average molecular size of less than 200 kDa.

16. The method of claim 10, wherein the sulfated hyaluronan has an average molecular size of less than 20 kDa.

17. The method of claim 10, wherein the sulfated hyaluronan has an average molecular size from 1 kDa to 10 kDa.

18. The method of claim 17, wherein (1) greater than 90% of the primary C-6 hydroxyl protons of the N-acetyl-glucosamine residue of the sulfated hyaluronan are substituted with a sulfate group, and (2) the sulfated hyaluronan has a degree of sulfation from 3.0 to 4.0.

19. The method of claim 17, wherein (1) 100% of the primary C-6 hydroxyl protons of the N-acetyl-glucosamine residue of the sulfated hyaluronan are substituted with a sulfate group, and (2) the sulfated hyaluronan has a degree of sulfation from 3.0 to 4.0.

20. The method of claim 10, wherein the pharmaceutically acceptable ester is a prodrug.

21. The method of any one of claims 1 to 9, wherein the sulfated glycosaminoglycan or the pharmaceutically acceptable salt or ester comprises at least one sulfate group and at least one primary C-6 hydroxyl position of an N-acetyl-glucosamine residue comprising an alkyl group or fluoroalkyl group.

22. The method of claim 21, wherein the alkyl groups is an unsubstituted alkyl group.

23. The method of claim 21, wherein the unsubstituted alkyl group is methyl.

24. The method of claim 21, wherein the fluoroalkyl group comprises at least one trifluoromethyl group.

25. The method of claim 21, wherein from 1% to 100% of the primary C-6 hydroxyl protons of the N-acetyl-glucosamine residue are substituted with an alkyl group or fluoroalkyl group.

26. The method of claim 21, wherein the modified hyaluronan has a molecular weight from 10 kDa to 2,000 kDa prior to modification.

27. The method of claim 21, wherein at least one C-2 hydroxyl proton and C-3 hydroxyl proton is substituted with a sulfate group.

28. The method of claim 21, wherein the modified hyaluronan is sulfated at the C-4 hydroxyl position of the N-acetyl glucosamine moiety, the C-2 position of the glucuronic acid moiety, the C-3 position of the glucuronic acid, or any combination thereof.

29. The method of claim 21, wherein the modified hyaluronan has a degree of sulfation from 0.5 to 4.0 per disaccharide unit.

30. The method of claim 21, wherein the alkyl group is methyl and at least one C-2 hydroxyl proton and/or C-3 hydroxyl proton is substituted with a sulfate group.

31. The method of claim 21, wherein the alkyl group is methyl, at least one C-2 hydroxyl proton and/or C-3 hydroxyl proton is substituted with a sulfate group, and the compound has a molecular weight of 2 kDa to 10 kDa.

32. The method of claim 21, wherein the sulfated glycosaminoglycan comprises
(a) a first modified hyaluronan or a pharmaceutically acceptable salt or ester thereof, wherein said first modified hyaluronan or its pharmaceutically acceptable salt or ester comprises (i) at least one primary C-6 hydroxyl proton of at least one N-acetyl-glucosamine residue substituted with a methyl group, (ii) an average molecular weight from 1 kDa to 15 kDa, (iii) a degree of methylation greater than 0 to 0.5 methyl groups per disaccharide unit; and (iv) a degree of sulfation of 2.5 to 4.0 sulfate groups per disaccharide unit; and
(b) a second modified hyaluronan or a pharmaceutically acceptable salt or ester thereof, wherein said second modified hyaluronan or its pharmaceutically acceptable salt or ester comprises (i) at least one primary C-6 hydroxyl proton of at least one N-acetyl-glucosamine residue substituted with a methyl group, (ii) an average molecular weight from 1 kDa to 15 kDa, (iii) a degree of methylation greater than 0 to 0.5 methyl groups per disaccharide unit; and a (iv) degree of sulfation of 2.5 to 4.0 sulfate groups per disaccharide unit, wherein pyridine is covalently bonded to the second modified hyaluronan or a pharmaceutically acceptable salt or ester thereof.

33. The method of claim 32, wherein the degree of methylation in the first and second modified hyaluronan is 0.03 to 0.3 methyl groups per disaccharide unit.

34. The method of claim 32, wherein the first and second modified hyaluronan has an average molecular weight from 1 kDa to 10 kDa.

35. The method of claim 32, wherein the degree of sulfation in the first and second modified hyaluronan is 3.0 to 4.0 sulfate groups per disaccharide unit.

36. The method of claim 32, wherein the amount of pyridine present is from 0.1 wt % to 4.0 wt % of the composition.

37. The method of claim 32, the degree of methylation in the first and second modified hyaluronan is 0.03 to 0.3 alkyl groups per disaccharide unit, the first and second modified hyaluronan has an average molecular weight from 1 kDa to 10 kDa, the degree of sulfation in the first and second modified hyaluronan is 3.0 to 4.0 sulfate groups per disaccharide unit, and the amount of pyridine present is from 0.1 wt % to 4.0 wt % of the composition.

38. The method of any one of claims 1 to 9, wherein the pharmaceutically acceptable salt comprises an organic salt, a metal salt, or a combination thereof.

39. The method of claim 38, wherein the pharmaceutically acceptable salt of the comprises a salt selected from the group consisting of NH₄⁺, Na⁺, Li⁺, K⁺, Ca⁺², Mg⁺², Fe⁺², Fe⁺³, Cu⁺², Al⁺³, Zn⁺², 2-trimethylethanolammonium cation (choline), or a quaternary salt of isopropylamine, trimethylamine, diethylamine, triethylamine, tripropylamine, ethanolamine, 2-dimethylaminoethanol, 2-diethylaminoethanol, lysine, arginine, and histidine.

40. The method of any one of claims 1 to 9, wherein the sulfated glycosaminoglycan comprises heparin sulfate or chondroitin sulfate.
